(19) United States
(12) Patent Application Publication
(10) Pub. No.: US 2008/0298549 A1
(43) Pub. Date: Dec. 4, 2008

(54) Web-Based Expert System for Educational Therapy Planning

(75) Inventors: Kathleen Ann Quill, Essex, MA (US); Cynthia Howroyd, Hanwell (CA)

Correspondence Address: LANG MICHENER, BCE PLACE, P.O. BOX 747, SUITE 2500, 181 BAY STREET, TORONTO, ON, M5J 2T7, CA

(73) Assignee: VIRTUAL EXPERT CLINICS INC., Fredericton, NB (CA)

(21) Appl. No.: 12/095,243

(22) PCT Filed: Nov. 30, 2006
(86) PCT No.: PCT/CA2006/001944

§ 371 Date: May 28, 2008

(30) Foreign Application Priority Data
Nov. 30, 2005 (CA) 2528466

(51) Int. Cl. A61N 5/10; A61B 5/00

(52) U.S. Cl. 378/65; 705/3

(57) ABSTRACT

The invention consists of an expert system supporting non-experts in their capacity to develop, implement, monitor and evolve therapy plans. Therapy plans in this application are educational and non-medical. The purpose of the therapy plan is to guide the caregiver or therapist in what to do for a client and how to do it. The client is a person with a previously diagnosed condition (psychological, cognitive, or physical) which responds to a teaching process. The plans are developed based on expert system analysis of user input. This analysis is reflective of decision making processes in the fields of both special needs education and rehabilitative medicine, including speech language pathology, psychology, physiotherapy, and occupational therapy.

### FIELD OF THE INVENTION

The present invention relates to the field of web-based expert systems for decision-support and management of complex processes. In particular, it relates to an expert system supporting non-experts in their capacity to develop, implement, and monitor therapy plans.

### BACKGROUND OF THE INVENTION

A growing concern for parents of children with developmental conditions, such as autism, is an inability to access the expertise necessary for proper educational therapy and optimal development of their child. Timely access is also a significant issue, as the earlier a child can receive therapy for a particular condition, the more likely the child will substantially benefit.

One of the reasons that access is limited is the need to provide on-going customization of the plan and ongoing training and guidance for those implementing the plan. In the case of early childhood therapy for autism, the therapy is likely to be intense (20 to 40 hours per week) and progress rapid (re-customization of the plan every few weeks), requiring a substantial and ongoing time investment of the supervising clinician in training, planning, and managing the efforts of the direct therapy staff and parents. In addition, a very high level of expertise is needed to provide quality guidance to intensive therapy programs for young, difficult-to-teach children with complex disorders such as autism. Therefore this expert guidance is both scarce and expensive. Parents who experience such barriers to access may engage in poor quality, ineffective therapy programs run by non-experts; they may not access therapy programs at all; or they may sit on therapy waiting lists for years during the most critical time for re-mediating the disorder or improving their child's development.

The opportunity exists to increase access to quality therapy through web-based expert technologies designed to capture the logic of therapeutic decision-making, and automate the on-going development and guidance necessary for educational therapy programs. In addition, the opportunity exists through e-learning technology to automate the just-in-time customized training needed to implement the plans.

Expert-based systems have been developed for medical use, and they are generally targeted towards medical diagnosis of conditions by physicians rather than educational and habilitative treatment by therapists (e.g. psychologist, speech language pathologist, occupational therapist, etc.). In those systems where medical treatment is suggested, there is no provision for ongoing developmental assessment of the client, nor is there any provision for user input concerning the treatment suggested. Also, many of these systems are designed for use by experts and are not accessible or useful for novice users. These systems fail to provide or increase access for those persons who need it the most, with the further acknowledgement that this is not their intent in the majority of systems.

Another consideration is the ability of the system to develop and evolve over time. The concept of tracking and analyzing collected data for trends (known as “data-mining”) has proven useful in many industries and is well-suited for use in computer database systems. While “data-mining” has seen some use in developing expert diagnostic systems, there are few developments in the areas of treatment and tracking of client progress. Generally, expert systems require regular maintenance and input of new data from specialists responsible for producing the system. Ideally, an expert system should be capable of evolving through data-mining in addition to receiving new input.

There is a need for an easily accessible expert system that is capable of collecting information from non-specialist (novice) users to generate a therapy plan as well as informing and allowing client caregivers to make choices about educational therapy options. This system can also include detailed instructions for novices in exercising educational therapy options, if necessary. There is a further need for an expert system capable of providing troubleshooting and plan review functions as well as ongoing progress and outcome measures that can be used by the individual user to refine a child's plans and by the expert system to automatically evolve.

### SUMMARY OF THE INVENTION

The invention is a web-based expert guidance method for improving habilitative conditions, consisting of stepping the user through two inter-dependent processes. The first process is to establish and evolve a curriculum or therapy plan consisting of goals or specific skills to work towards and procedures or activities to follow for achieving each goal. The second process is to teach the caregiver or therapist the competencies and recommend the techniques necessary for effectively implementing the curriculum or in general helping the client.

To establish the curriculum the system first analyses general user input to create an overall profile of a client diagnosed with a condition, such as an autistic child, and a profile of the therapist, educator, or caregiver who will be implementing the therapy plan. These profiles are applied in conjunction with the system's clinical model of priorities to create recommendations on what treatment strategies and therapy objectives should be included in a therapy plan.

Second, user input is collected from one or more users to create a skills analysis detailing specific patterns of strengths and weakness in the client. Based on the skills analysis, the expert algorithm determines a set of priority-ranked specific skills or goals to work towards in therapy.

Third, based on the subset of therapy goals and prior user inputs, the algorithm recommends a set of activity procedures to carry out with the client to achieve each specific goal.

As the therapy plan is implemented, the system tracks client progress through repeated skills re-assessments. When targeted skills have been attained, the system prompts the user to update the therapy plan for the client, and the three step process of curriculum development is repeated.

The core process of developing and implementing therapy plans is complemented by a multi-part training and guidance mechanism as an additional means of quality assurance.

First, based on user inputs, a ‘strategy plan’ is provided which recommends to the user instructional techniques to follow while carrying out the activity procedure with the client. Second, based on user inputs, a ‘support plan’ is provided which recommends supports to use with the client/child to prevent problems and compensate for deficits in abilities. Both the strategy and support plans include multimedia training for each recommended technique or strategy including text-based explanations and possibly video vignettes or graphics. Third, a multimedia encyclopedia also provides support for all difficult-to-understand terms on a screen-by-screen basis. Fourth, a troubleshooting mechanism helps guide the user in what to do when experiencing problems or having questions. Fifth, each module is supported by e-learning materials and frequently asked questions. That is each step the user takes while setting up the intervention plan, evolving the intervention plan, and maintaining the intervention plan is supported by expert knowledge already embedded in the system.

If attainment in skills is not achieved over a reasonable period of time the system recommends remedial action which may involve review and recommended changes to the curriculum, support plan, or strategy plan.

The system also provides the user with the knowledge and ability to log and track progress for the child in order to continue to properly evolve the plan based on the child's current abilities.

A primary advantage of the invention is that novice users, such as the parents of an autistic child, can gain access to expert driven knowledge, specialized therapy plans, support and guidance for implementing those plans. This level of access can be invaluable for those who are otherwise restricted from accessing services and or specialists due to financial or geographic limitations.

Another advantage is gained through analyzing the progress and therapy plan data collected by the system. The expert algorithm can be refined and improved through the ongoing use of the system, reducing the time and effort required from specialists to monitor and maintain the system.

Other and further advantages and features of the invention will be apparent to those skilled in the art from the following detailed description thereof, taken in conjunction with the accompanying drawings.

### BRIEF DESCRIPTION OF THE DRAWINGS

The invention will now be described in more detail, by way of example only, with reference to the accompanying drawings, in which like numbers refer to like elements, wherein:

FIG. 1 is a flowchart showing the steps of the present invention;

FIG. 2 is a flowchart of the overall process;

FIG. 3 is a flowchart of the support and strategy plan development process;

FIG. 4 is a flowchart of the curriculum development process;

FIG. 5 is a flowchart of the registration process;

FIG. 6 is a flowchart of the Setup/First Time user “wizard” process;

FIG. 7 is a flowchart of the Intervention process;

FIG. 8 is a flowchart of the Development Strategy and Support process;

FIG. 9 is a flowchart of the Curriculum Plan/Intervention Plan Execution process;

FIG. 10 is a flowchart of the progress viewing process;

FIG. 11 is a flowchart of the curriculum update process;

FIG. 12 is a flowchart of the support and embedded knowledgebase process.

### DETAILED DESCRIPTION OF THE PREFERRED EMBODIMENTS

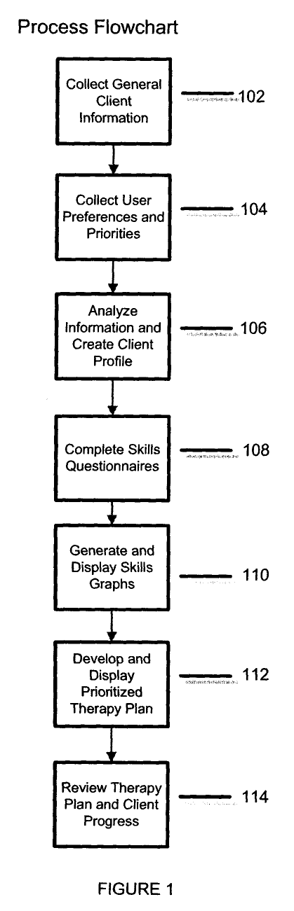

The invention consists of a series of steps, as shown in FIG. 1, that are performed by a user through a web-based interface and which occur in a repetitive cycle over time. The first two steps are designed to gather general profile information about the client (child/patient) and about the parent or therapist (user) using the system.

The initial client profiling (step 102) involves the user inputting general case information and answering questions that provide the system with information on client clinical type. The client's case information such as age and hours in therapy are obtained through a fill-in-the-blank form. The client's severity of diagnosis and clinical type is ascertained by scenario-based multiple choice selection and a ranked score-based questionnaire.

The second step (step 104) involves capturing user methodological preferences and priorities by requiring selection of a limited set of scenario-based options (e.g. “you like to work in a structured setting . . . ”) or a combination thereof, and by using ranked scoring—based questionnaires (e.g. “You are most concerned about your child's relationship with others”).

The next step (step 106) in the process after the general profiling (104) involves a careful analysis of the client's relative strengths and weaknesses and determining specific, prioritized areas of need for therapeutic focus. This needs analysis involves three steps.

The first step (step 106) is to present and recommend a prioritized set of skill types for therapy based on the general profiling analysis generated by the algorithm. The user is permitted to alter skill type selections such as motor, social, behaviour etc., to reflect unique therapist recommendations, special priorities (e.g. areas of weakness most stressful to the parent), or exceptional circumstances (e.g. blindness).

The next step (step 108) is to complete a skills questionnaire for each skill category. The user and/or invited participants are to complete these relevant questionnaires. Questionnaires are designed as a series of assessment questions (e.g. “does the child respond when spoken to?”), typically using a ranking system or multiple-choice series. In the present embodiment, the client is an autistic child, with the questions being posed either to a parent, teacher, paraprofessional, or therapist.

The last step (step 110) is to take the completed questionnaires and the responses to each skill type are processed by an expert algorithm to generate a skills graph. The client skills graph for that skill category is then displayed to the user with an explanation of terms.

The system uses easy-to-understand questions and supporting examples in order to allow novices or non-experts to complete the questionnaires. The result is that the system can provide treatment plans in situations where access to such specialists is not possible.

Client confidentiality is protected by a variety of measures including: a password-based access system, lack of storage of credit card information, and optional use of the child's full name or real name and picture. When an appropriate party (typically a parent) registers a child, they will be provided with a series of user IDs and passwords for that child. The other accounts can then be assigned to those persons who will be invited to help complete the questionnaire process or implement the therapy plan. However, control of the IDs remains with the original party, who can revoke and reassign user IDs as necessary (i.e. if the child's teacher is changed by a change in schools).

With the above steps completed, the expert algorithm is used to develop (step 112) a therapy plan consisting of prioritized client goals as well as activities for achieving these goals. The user may edit the therapy plan in a number of ways (dependent on user knowledge level), including deleting or adding therapy goals.

The user may then periodically review and modify (step 114) the responses to questions as the client progresses and thereby update both the skills graph and the therapy plan.

Users are educated around options for choosing instructional methodology including overall approach (method selection), problem-solving strategies (support plan), and teaching techniques (strategy plan). By clicking on an option, the user is provided with more information about that option, including definitions, explanations and illustrations (text, image, and/or audio/video) to aid in choice-making and eventual implementation. Specific options are recommended based on prior user inputs.

While methodological recommendations are provided by the expert algorithm, the user may choose to select options independently.

Additionally, the user may self-identify a skill level for training (phase one, two, three) which can be used to change the strategy or support method suggested. For example, a parent may be given a basic strategy to perform with their child, while a professional therapist would learn a more complex set of strategies suitable to their expertise.

This system also allows the user to print curriculum plans for future use and reference. Materials for curriculum and instruction methodology are available in the same fashion.

As part of the final steps (112, 114), once a profile is established and a plan has been adopted and followed, the client profile is updated at regular intervals, such as every six months. At that time, the user(s) will update the child's progress and a revised profile will be generated. Results can be compared to the past to determine if the client has progressed and improved. A new therapy plan is also generated by the algorithm using all available profiles. Progress and improvement (or lack thereof) may result in prompted changes to the priorities of the different goals and of the different methods available to achieve goals.

As time goes on, the individual client's progress will be tracked throughout the curriculum plan (as activities and objectives are completed) and will be recorded in the profile. The results for each client in terms of development using various methods and starting from various profiles can then be used to further enhance the algorithm used to prioritize goals and methods without compromising client confidentiality. In this way, methods that prove particularly successful (or unsuccessful) in achieving certain goals with certain clients (based on profile) can be prioritized accordingly, improving the capabilities of the expert algorithm over time.

Given the volume of information contained in the system, as well as the necessity for up-to-date resources and information to be input into the expert system algorithm, the system can serve a secondary purpose of providing access to relevant information collected by the system. Users can search and peruse articles, research papers, clinical studies and treatment methods that are necessarily collected as part of maintaining the expert system.

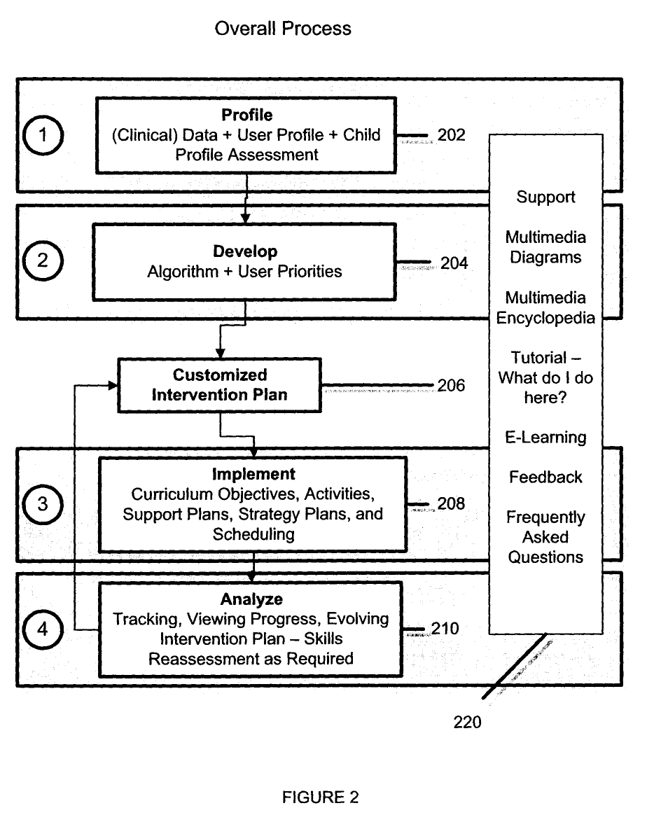

FIG. 2 illustrates the stepwise process described above as shown in a facsimile screenshot of a front page for the web-based system. The steps in the process as shown: Profile (step 202), Develop (step 204), Implement (step 208) and Analyze (step 210) in sequence along with a summary of the processes at each step. The Customized Intervention Plan (step 206) is located at the center of the other steps for easy reference and access. A sidebar (220) is also provided to link to various support and help references associated with the web-based system.

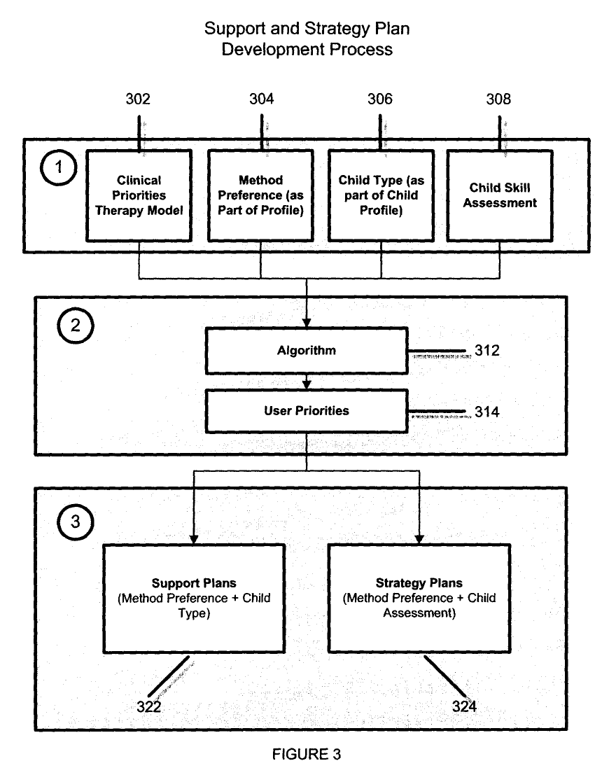

FIG. 3 illustrates the support and strategy plan development process. The first step involves combining the information gained through the profiling process. The clinical priorities (302), method preferences (304), child type (306) and child skill assessment (308) are taken and passed through the system algorithm (step 312) which, when combined with the user priorities (step 314) produces a set of support plans (322) and strategy plans (324) for implementation.

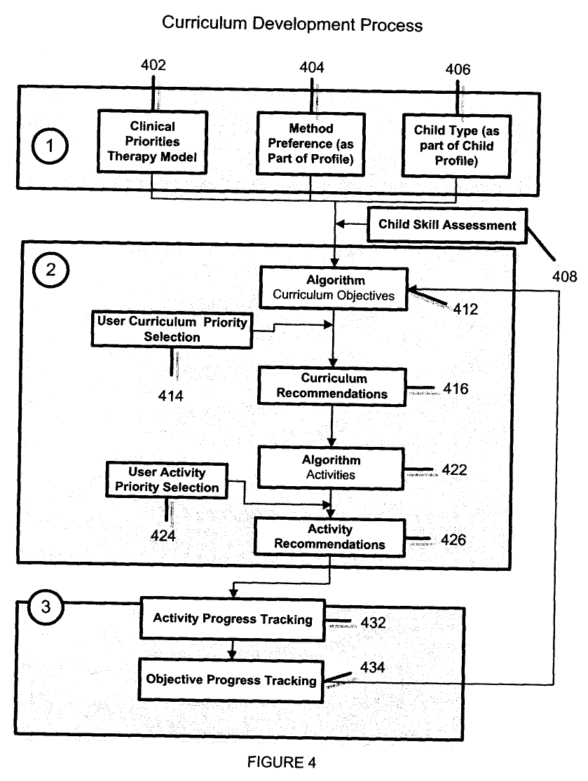

FIG. 4 illustrates the curriculum development process. The three profile components: clinical priorities (402), user method preference (404) and child type (406) are taken and combined with the child skill assessment (408) and fed into the system curriculum algorithm (412). The algorithm produces a result, which is assessed against the user curriculum priorities (414) and a set of curriculum recommendations (416) is produced. Similarly, the information, along with the curriculum recommendations, is fed into the activity algorithm (422) and the result produced is assessed against the user activity priorities (424) and a set of activity recommendations (426) is produced. From this point, the activity progress tracking (432) and objective progress tracking (434) modules are engaged and the feedback from those modules is fed back into the curriculum algorithm (412).

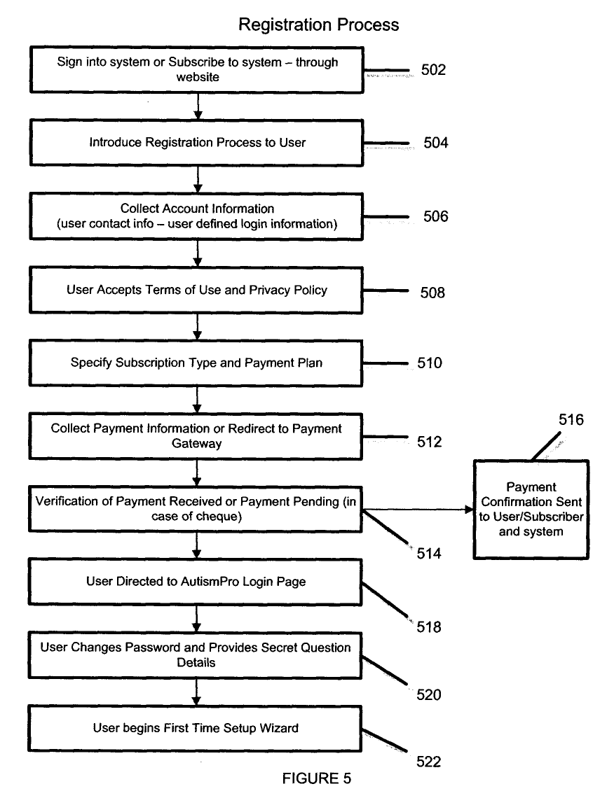

FIG. 5 illustrates the user/client registration process. The user signs into the system (step 502), preferably through a website and the registration process is presented (step 504). The user account information is collected (step 506) including contact and login information. After accepting the terms of use and privacy policy for the system (step 508), the user selects a subscription type and appropriate payment plan (step 510). Once a payment plan is selected, the appropriate payment information is collected or the user is redirected to a payment provider gateway (step 512). Once payment is verified, or confirmed as pending (step 514) the user is directed to the login page (step 518). For a payment that requires further confirmation, such as a cheque, a confirmation can be sent (step 516) to the user and the system.

Once logged in, the user is provided (step 520) with an opportunity to select a password and proceed with other security measures, such as a ‘secret question’ for lost password retrieval. Then, the user is able to access the system, beginning (step 522) with the first time user setup process.

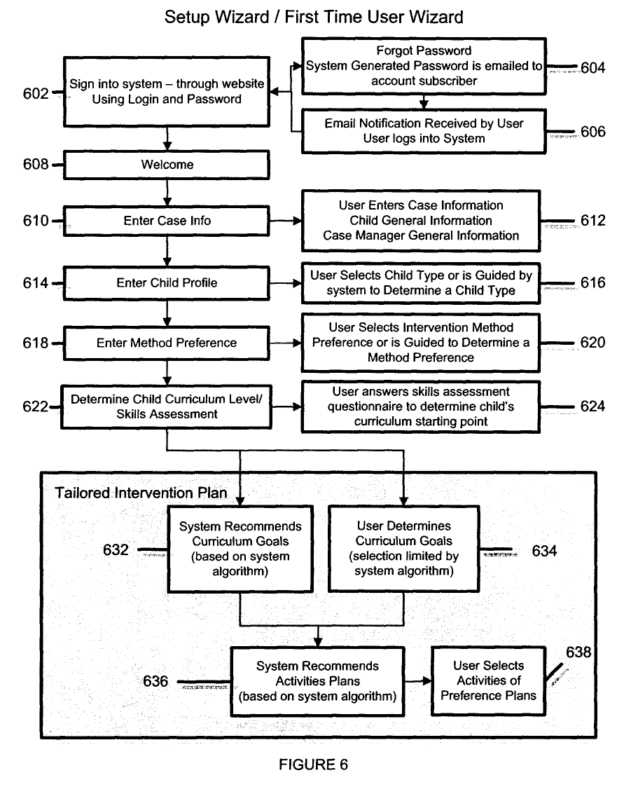

FIG. 6 illustrates the first-time user setup process as a “wizard”. The user signs in to the system (step 602) with their login and password. If the user has forgotten their password, a new one can be generated by the system (step 604) and emailed to the user (step 606) at the contact information provided by the user.

Once logged in, the user is presented with a welcome screen (step 608) and asked to enter the case information (step 610). The information entered will vary depending on the identity of the user (612). Once the case information is entered, next the child profile is entered (step 614). The profile can be selected by the user independently or with guidance from the system (616). Then, the user selects their intervention method preference (step 618), again with guidance from the system as necessary (620).

Once the preference information is input, the child skills assessment is conducted (step 622). The user responds to a questionnaire to create a starting point for the child's skill and development (624), as discussed above. Once this assessment is complete, an intervention plan, with curriculum and activities, can be developed.

The intervention plan is developed by taking curriculum goals recommended by the system (step 632) and those determined by the user (step 634) and the system using that information to develop recommended activity plans (step 636) which are then selected by the user (step 638) according to their preferences.

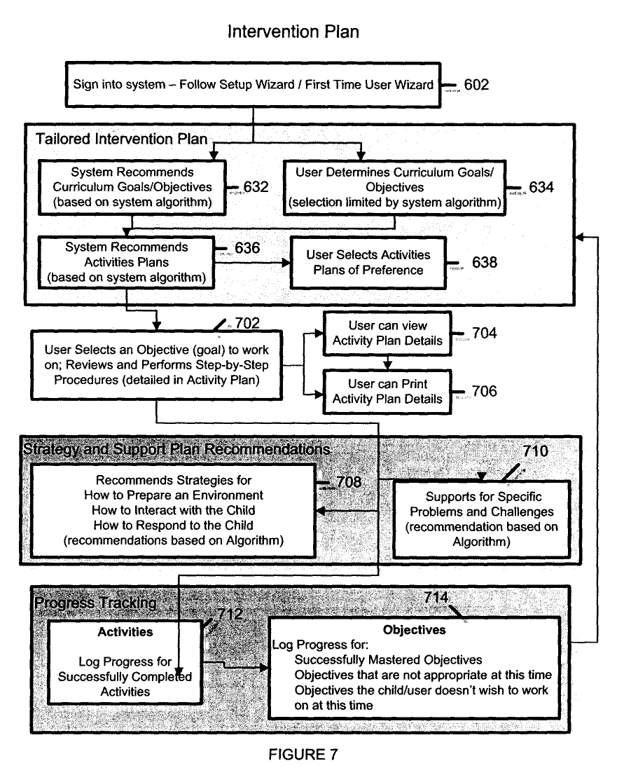

FIG. 7 illustrates the intervention plan process. Once the activity plans (636) are developed, the user selects (step 702) a goal, and is provided with the procedures and activities for use in achieving the goal. The details can be viewed (step 704) and printed (step 706) by the user for use.

As part of the intervention plan, the system further recommends strategies (step 708) and supports (step 710) to assist in pursuing the activity plan. Furthermore, the system enables progress tracking of activities (step 712) to note successful completion, and objectives (step 714) to enable the user to track what is being achieved, what is working, and what is not.

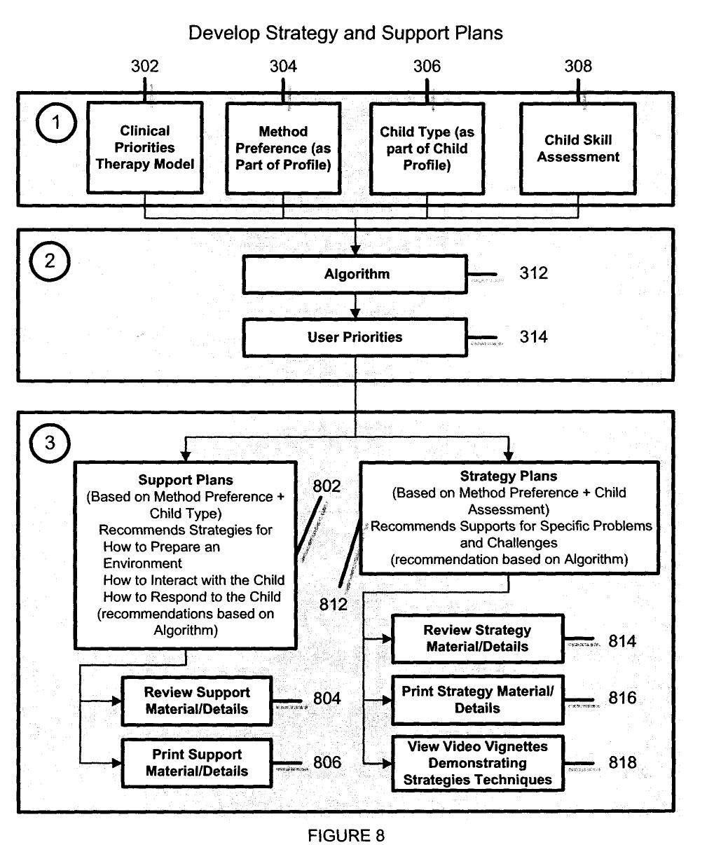

FIG. 8 illustrates the strategy and support process. Taking the information from the system algorithm (312) and the user-defined priorities (314), the system is then able to develop support plans (step 802) and strategy plans (step 812) to assist in execution of the intervention plan. The support plans (802) set out strategies for preparing the environment and the child for the activity and how to interact and respond to the child during the activity. The material can be reviewed (step 804) and/or printed (step 806) by the user as necessary. The strategy plans (812) are designed to assist in addressing specific problems or challenges that may arise as part of the activity. In addition to being able to review (step 814) and print (step 816) the strategies, the strategy plans preferably further include audio/video demonstrations (step 818) for specific assistance with specific exercises.

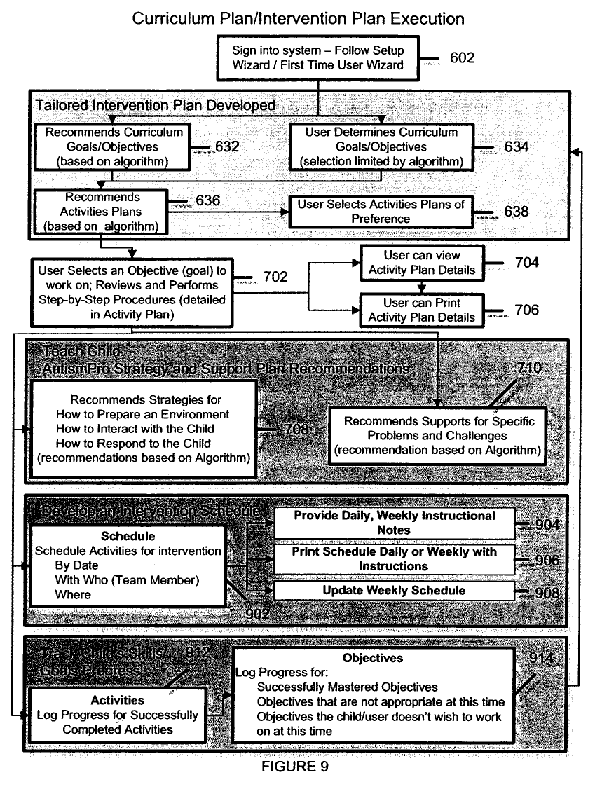

FIG. 9 illustrates the plan execution process for the curriculum plan and the intervention plan. As part of the process, once the user has selected an objective (702), an intervention schedule is developed (step 902) for use in reaching the objective. The schedule provides daily and/or weekly instructional notes (904), with the ability to print sections for reference as required (906), and the ability to update the schedule (908) as necessary. Additionally, completed activities are tracked (step 912) along with information about completed objectives, and objectives that have been excluded at the current stage of treatment (step 914).

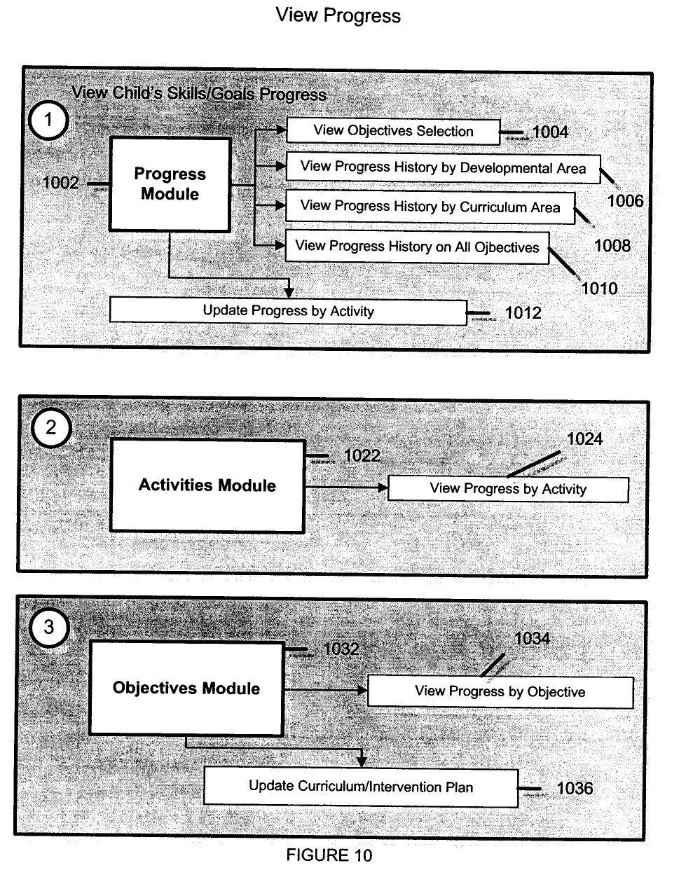

FIG. 10 illustrates the progress report and viewing process. The progress module (1002) is accessed and different aspects of the treatment plan can be reviewed. The current objectives (1004), progress by developmental area (1006), progress by curriculum area (1008) and progress by objectives (1010) are all demonstrative of the type of progress information that can be reviewed. The module can also be updated (step 1012) as necessary according to the activity. The module may be further broken down into an activity module (1022) to allow for review of progress by activity (1024) and an objectives module (1032) to allow for review of progress by objective. The objectives module may addition allow for updating (step 1036) according to the curriculum and intervention plans.

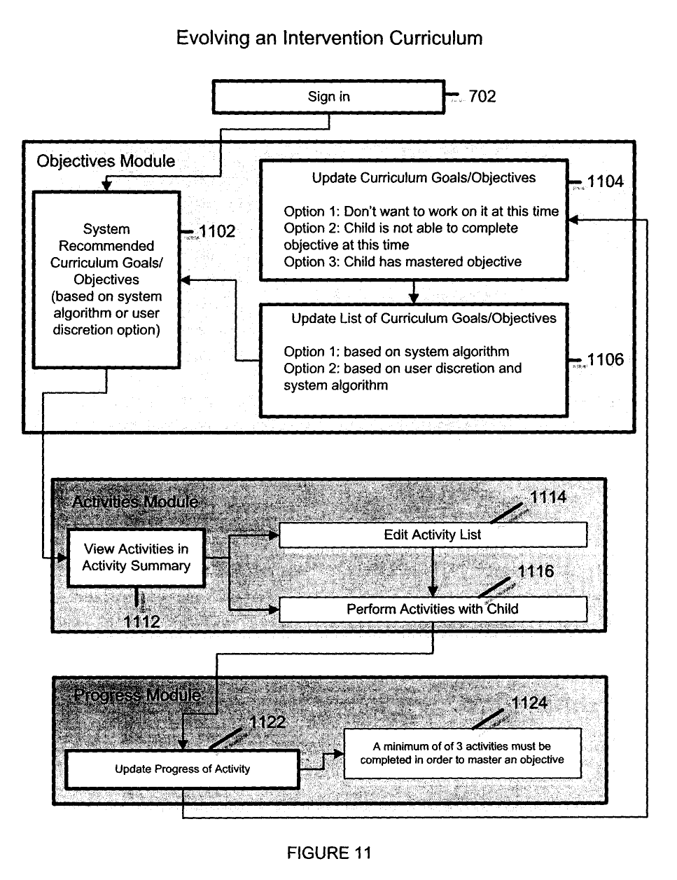

FIG. 11 illustrates the curriculum updating process. Once signed in (702) the user reviews the recommended curriculum goals and objectives (step 1102). A summary of current activities is displayed (step 1112) and can be edited (step 1114) or activity performance recorded (step 1116). The progress of the activity is updated step 1122) and progress towards objective completion is assessed (step 1124). This information is then used to update the curriculum goals and objectives (step 1104) and an updated list is created (step 1106) and presented for review by the user (step 1102).

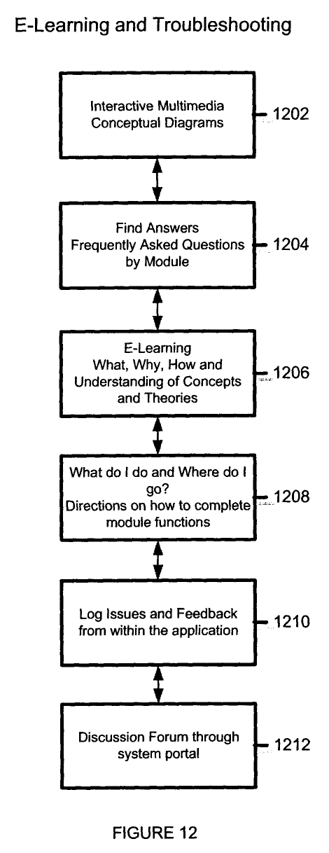

FIG. 12 illustrates the support and knowledge base access process. A selection of some of the potential choices is shown. Demonstration materials, such as interactive multimedia diagrams (1202) and links to different modules (1208) may be provided. An e-learning center (1206) to provide information and further understanding of the theories behind the system, as well as a discussion forum (1212) are useful in furthering progress and development of treatment theories. Lastly, basic website assistance, such as a frequently asked question (FAQ) database (1204) and feedback/issue reporting link (1210) further application development.

This concludes the description of a presently preferred embodiment of the invention. The foregoing description has been presented for the purpose of illustration and is not intended to be exhaustive or to limit the invention to the precise form disclosed. Many modifications and variations are possible in light of the above teaching and will be apparent to those skilled in the art. It is intended the scope of the invention be limited not by this description but by the claims that follow.

What is claimed is:

1. An automated expert system-based method for planning, tailoring and evolving therapy programs using a web or wireless delivery system, comprising:
a) collecting user input from one or more users concerning a client diagnosed with a medical or psychological condition amenable to an educational therapy program and concerning preferences and priorities of persons responsible for said client, said persons including: parents, caregivers, therapists, educators;
b) analyzing said user input using an expert algorithm to create a client profile;
c) providing a treatment plan based on said client profile, said treatment plan consisting of treatment activities and a set of priority-ranked therapy goals, said priority ranking determined by said expert algorithm; and
d) repeating steps a) to c) on an ongoing basis to track client progress and provide updated treatment plans for said client.

2. The method of claim 1, wherein said treatment plan includes a combination of support plans for preparing and introducing activities and strategy plans for conducting and practicing activities.

3. The method of claim 2, wherein said strategy plans further include multimedia demonstrations of activities.

4. The method of claim 1, wherein said user is capable of overriding rankings determined by said expert algorithm.

5. The method of claim 1, further including a step e) of analyzing client progress, feedback, and treatment plans for multiple clients to develop improvements to said expert algorithm.

6. An automated expert system for planning, tailoring and evolving therapy programs, comprising:
a) a collection unit for collecting user input from one or more users concerning a client diagnosed with a medical or psychological condition amenable to an educational therapy program and concerning preferences and priorities of persons responsible for said client, said persons including: parents, caregivers, therapists, educators;
b) an analysis unit for analyzing said user input using an expert algorithm to create a client profile and for providing a treatment plan based on said client profile, said treatment plan consisting of treatment activities and a set of priority-ranked developmental goals, said priority ranking determined by said expert algorithm; and
d) a tracking unit, operating on an ongoing basis to track client progress and provide updated treatment plans for said client.

7. The system of claim 6, further including a database unit for storing articles, media and other related information for access by users of the system.

8. The system of claim 6, further including a library of demonstration media, including audio and visual media, for demonstrating and assisting the user in conducting planned activities.

9. The system of claim 6, wherein said system is implementable as a web-based client-server system.

10. The system of claim 6, further including a data-mining unit for analyzing client progress, feedback, and treatment plans for multiple clients to develop improvements to said expert algorithm.
